(19) United States
(12) Patent Application Publication
(10) Pub. No.: US 2026/0248725 A1
(43) Pub. Date: Aug. 27, 2026

(54) BIOMINERAL CRYSTAL MATERIAL, PREPARATION METHOD THEREOF AND SLEEP-AIDING TECHNOLOGY PILLOW

(71) Applicant: ZHENGQI NEW MATERIALS TECHNOLOGY(CHANGZHOU) CO., LTD., Changzhou (CN)

(72) Inventors: LEI GUO, Changzhou (CN); BOWEN GUO, Changzhou (CN)

(73) Assignee: ZHENGQI NEW MATERIALS TECHNOLOGY(CHANGZHOU) CO., LTD., Changzhou (CN)

(21) Appl. No.: 19/653,081

(22) Filed: Apr. 20, 2026

Related U.S. Application Data
(63) Continuation of application No. PCT/CN2025/116765, filed on Aug. 25, 2025.

(30) Foreign Application Priority Data
Jul. 1, 2024 (CN) 2024108690738

(51) Int. Cl. A61K 9/16; A47G 9/10; A61K 9/00; A61K 36/282; A61K 36/53; A61K 36/61; A61K 36/738; A61P 25/20

(52) U.S. Cl. CPC A61K 9/1611; A61K 9/007; A61K 36/282; A61K 36/53; A61K 36/61; A61K 36/738; A61P 25/20; A47G 9/10

(57) ABSTRACT

Provided are a biomineral crystal material, a preparation method thereof and a sleep-aiding technology pillow, which belong to the technical field of material science. The method includes: modifying a surface of natural mineral crystal, mixing the modified natural mineral crystal with TiO₂/Fe₃O₄ doped intercalated montmorillonite to prepare TiO₂/Fe₃O₄ doped intercalated montmorillonite modified crystal powder, then modifying the surface of the powder with polydopamine, adsorbing aromatic essential oil onto the powder, and embedding the powder with sodium alginate to prepare a spherical biomineral crystal material. The sleep-aiding technology pillow prepared from the biomineral crystal material prepared by this disclosure can slowly release essential oil molecules to improve service durability, and the spherical particles thereof can also facilitate acupoint stimulation to help improve sleep quality.

### CROSS-REFERENCE TO RELATED APPLICATIONS

The application claims priority to Chinese patent application No. 2024108690738, filed on Jul. 1, 2024, the entire contents of which are incorporated herein by reference.

### TECHNICAL FIELD

This disclosure relates to the technical field of material science, and in particular, to a biomineral crystal material, a preparation method thereof, and a sleep-aiding technology pillow.

### BACKGROUND

With the continuous improvement of living standards, people are paying growing attention to hygienic, safe and health-beneficial products. An increasing number of people suffer from poor sleep quality or even all-night insomnia, as they feel stressed from the accelerated pace of modern life. A large number of people also develop neurasthenia as a result of poor sleep quality and insomnia, and accordingly, sleep-aiding pillows have emerged as required.

Natural crystal is a quartz crystal mineral, its main chemical component is silicon dioxide with the chemical formula of SiO₂, and it is also known as water essence, water jasper or water jade. Well-developed single crystals of natural crystal are hexagonal pyramids, so natural crystal usually exists as a massive or granular aggregate. Natural crystal can affect human health by generating electromagnetic fields, regulating the human body's energy field and other means, which is the so-called “mineral therapy”. In addition, natural crystal can collect negative ions from the outside and release them into the surrounding environment, thereby improving indoor air quality and relieving symptoms such as headache and insomnia.

However, directly adding natural crystal into pillows cannot achieve a desirable sleep-aiding effect, so it is necessary to modify the natural crystal.

Chinese Patent Publication No. CN110101268A discloses a filler for a sleep-aiding pillow and a sleep-aiding pillow. By mixing Artemisia argyi, Trachelospermum jasminoides, Phlomis umbrosa, Lavender and Chrysanthemum, the advantages of each component are fully utilized, which can effectively alleviate insomnia, aid sleep, help people with dreaminess, easy waking, difficulty falling asleep, sleep discomfort and other conditions to soothe nerves and relieve fatigue, and achieve an ideal deep sleep state. However, when traditional Chinese medicine components are directly placed in a pillow, the effective components are released quickly, which shortens the effective service life, and meanwhile causes discomfort to people due to excessively high odor concentration in a short period of time.

### SUMMARY

An objective of this disclosure is to provide a biomineral crystal material, a preparation method thereof, and a sleep-aiding technology pillow. The biomineral crystal material can slowly release essential oil molecules to improve service durability, and the spherical particles thereof can also facilitate acupoint stimulation to help improve sleep quality. Meanwhile, by releasing infrared waves, forming a magnetic field, and releasing negative ions into the surrounding environment, the material can purify organic pollutants, formaldehyde and the like in the air, improve indoor air quality, and relieve symptoms such as headache and insomnia, which has broad application prospects.

The technical solution of this disclosure is realized as follows:
this disclosure provides a preparation method of the biomineral crystal material; the method includes: modifying a surface of natural mineral crystal, mixing the modified natural mineral crystal with TiO₂/Fe₃O₄ doped intercalated montmorillonite to prepare TiO₂/Fe₃O₄ doped intercalated montmorillonite modified crystal powder, then modifying the surface of the powder with polydopamine, adsorbing aromatic essential oil onto the powder, and embedding the powder with sodium alginate to prepare a spherical biomineral crystal material.

As a further improvement of this disclosure, the method includes the following steps:
S1. Surface modification of natural mineral crystal: ball-milling natural mineral crystal, adding the milled crystal into ethanol, adding a silane coupling agent, heating the mixture and carrying out reaction under stirring, filtering, washing, and drying to prepare surface-modified natural mineral crystal powder;
S2. Preparation of TiO₂/Fe₃O₄ doped intercalated montmorillonite modified crystal powder: adding tetrabutyl titanate into ethanol to form a solution A, dissolving melamine, ferric chloride and ferrous chloride in water to obtain a solution B, dropwise adding the solution B into the solution A, stirring evenly to obtain a pillaring agent, dropwise adding the pillaring agent into a montmorillonite suspension while dropwise adding ammonia water, adding the surface-modified natural mineral crystal powder prepared in step S1, heating the mixture and carrying out reaction under stirring, standing for aging, and performing microwave heating and drying, washing, ball milling and calcination to prepare the TiO₂/Fe₃O₄ doped intercalated montmorillonite modified crystal powder;
S3. Preparation of modified crystal powder: adding the TiO₂/Fe₃O₄ doped intercalated montmorillonite modified crystal powder prepared in step S2 into water, adding dopamine hydrochloride and a catalyst, heating the mixture and carrying out reaction under stirring, filtering, washing, and drying to prepare the modified crystal powder;
S4. Essential oil adsorption: adding the modified crystal powder prepared in step S3 into petroleum ether, adding compound aromatic essential oil, and evaporating the solvent to dryness to prepare essential oil-adsorbed modified crystal powder; and
S5. Preparation of biomineral crystal material: adding the essential oil-adsorbed modified crystal powder prepared in step S4 into water, adding sodium alginate and an emulsifier to obtain an aqueous phase, dropwise adding the aqueous phase into fish oil for emulsification, then dropwise adding a calcium chloride solution, performing room temperature curing, filtering, washing and drying to prepare the biomineral crystal material.

As a further improvement of this disclosure, in step S1, the ball milling is performed for 0.5-1.5 h, the mass ratio of the natural mineral crystal to the silane coupling agent is 50:4-7, the silane coupling agent is at least one selected from a group consisting of KH550, KH560, KH580, KH590, KH602 and KH792, and the mixture is heated and carried out reaction under stirring at 40-50° C. for 1-3 h.

Preferably, the silane coupling agent is a mixture of KH550 and KH580 with a mass ratio of 7-10:3-5.

As a further improvement of this disclosure, in step S2, a mass ratio of tetrabutyl titanate, melamine, ferric chloride, ferrous chloride, montmorillonite to the surface-modified natural mineral crystal powder is 5-7:0.5-1:3.4-3.5:1.2-1.3:30-50:100, the mixture is heated and carried out reaction under stirring at 65-75° C. for 2-4 h, the ball milling is performed for 1-2 h, and the calcination is performed at 450-550° C. for 1-3 h.

As a further improvement of this disclosure, in step S3, a mass ratio of the TiO₂/Fe₃O₄ doped intercalated montmorillonite modified crystal powder, the dopamine hydrochloride to the catalyst is 20-30:14-18:1-2, the mixture is heated and carried out reaction under stirring at 40-50° C. for 2-3 h, and the catalyst is a Tris-HCl solution with pH=8.5-9.

As a further improvement of this disclosure, in step S4, the mass ratio of the modified crystal powder to the compound aromatic essential oil is 100:15-22, the compound aromatic essential oil is at least two selected from a group consisting of lemon essential oil, bergamot essential oil, rose essential oil, clove essential oil, lavender essential oil and wormwood essential oil, and the essential oil is prepared by subjecting raw materials to supercritical fluid extraction.

Preferably, the compound aromatic essential oil is a mixture of the rose essential oil and the wormwood essential oil with a mass ratio of 5-7:3-4.

As a further improvement of this disclosure, in step S5, a mass ratio of the essential oil-adsorbed modified crystal powder, the sodium alginate to the emulsifier is 10-13:17-20:1-2, the emulsifier is at least one selected from a group consisting of Tween-20, Tween-40, Tween-60, Tween-80 and Tween-85, and the room temperature curing is performed for 20-30 min.

This disclosure further protects a biomineral crystal material prepared by the above preparation method.

This disclosure further protects an application of the above biomineral crystal material in preparing sleep-aiding products.

This disclosure further provides a sleep-aiding technology pillow, where a pillow core thereof is filled with the above biomineral crystal material at a filling amount of 0.01-100 wt %.

This disclosure has the following beneficial effects:
the natural mineral crystal is used in this disclosure, and the natural crystal can affect human health by generating electromagnetic fields, regulating the human body's energy field and other means, which is the so-called “mineral therapy”. In addition, natural mineral crystal can collect negative ions from the outside and release them into the surrounding environment, thereby improving indoor air quality and relieving symptoms such as headache and insomnia. Meanwhile, the natural mineral crystal is modified by a silane coupling agent, and the modification with KH550 and KH580 enables the surface of the crystal powder to carry sulfhydryl and amino groups, which can form hydrogen bonds and Van der Waals force interactions with groups such as hydroxyl groups on the surface of montmorillonite, so that the montmorillonite is fixed on the surface of the crystal powder.

In the preparation of the TiO₂/Fe₃O₄ doped intercalated montmorillonite, anatase TiO₂ not only has excellent adsorption performance, but also has superior photocatalytic performance. Under the doping effect of melamine, TiO₂ is doped with N and C atoms, and meanwhile, the sulfhydryl groups of the silane coupling agent also dope S elements into TiO₂, thereby changing the electron transition energy level of TiO₂, enabling it to perform photocatalysis within the wavelength range of visible light, and achieving excellent effects of degrading organic pollutants, purifying formaldehyde, resisting bacteria and mites, etc. In addition, the magnetic Fe₃O₄ can also form a magnetic field and emit infrared waves, so as to achieve the effects of magnetotherapy and infrared wave soothing for the human body during sleep. Meanwhile, the interlayer spacing of the montmorillonite intercalated with TiO₂/Fe₃O₄ dopant is increased, which not only maintains the lamellar structure before intercalation, but also presents layer exfoliation in the structure, thus increasing the specific surface area of the montmorillonite, further helping to improve its adsorption performance, facilitating the purification of ambient air, increasing the adsorption rate of aromatic essential oil, and enhancing the sustained release effect of the adsorbed aromatic essential oil.

This disclosure further modifies the surface of the prepared TiO₂/Fe₃O₄ doped intercalated montmorillonite modified crystal powder with polydopamine, so that the surface thereof carries abundant active groups such as hydroxyl groups, amino groups and carboxyl groups, which can generate a variety of interaction forces with aromatic essential oil molecules, improve the adsorption of essential oil molecules, thereby enhancing the sustained release effect of the essential oil, and further improving the sleep-aiding effect of the essential oil and its service durability.

The rose essential oil and wormwood essential oil adsorbed in this disclosure not only have a good sleep-aiding effect, but also can achieve antibacterial, mothproof, anti-mildew and anti-mite effects, improve the service durability of the technology pillow, and have a synergistic effect.

This disclosure embeds the prepared essential oil-adsorbed modified crystal powder with sodium alginate to prepare spherical embedded particles. On one hand, the spherical particles enable the slow release of essential oil and improve the service durability; on the other hand, the spherical particles can also facilitate acupoint stimulation to help improve sleep quality.

The sleep-aiding technology pillow prepared from the biomineral crystal material of this disclosure can slowly release essential oil molecules to improve service durability, and its spherical particles can also facilitate acupoint stimulation to help improve sleep quality. Meanwhile, by releasing infrared waves, forming a magnetic field, and releasing negative ions into the surrounding environment, the material can purify organic pollutants, formaldehyde and the like in the air, improve indoor air quality, and relieve symptoms such as headache and insomnia, which has broad application prospects.

### DETAILED DESCRIPTION OF THE EMBODIMENTS

The technical solution in the embodiments of this disclosure will be clearly and completely described below. Apparently, the described embodiments are only some rather than all of the embodiments of this disclosure. Based on the embodiments of this disclosure, all other embodiments obtained by a person skilled in the art without creative work shall fall within the protection scope of this disclosure.

The rose essential oil and the wormwood essential oil are prepared by subjecting rose and wormwood to supercritical fluid extraction, respectively.

The natural mineral crystal is a mixed ore containing natural mineral crystal and rare earth concentrate with a mass ratio of 10:5. The natural mineral crystal is purchased from Lianyungang Haisheng Quartz Technology Co., Ltd., and the rare earth concentrate is purchased from China Northern Rare Earth (Group) High-Tech Co., Ltd.

### Example 1

This example provides a preparation method of a biomineral crystal material, which specifically includes the following steps:
S1. Surface modification of natural mineral crystal: 50 parts by weight of natural mineral crystal were ball-milled for 0.5 h, the milled crystal was added into 200 parts by weight of ethanol, 4 parts by weight of silane coupling agent was added, the mixture was heated to 40° C. and carried out reaction under stirring for 1 h, then filtered, washed and dried to prepare the surface-modified natural mineral crystal powder;
the silane coupling agent is a mixture of KH550 and KH580 with a mass ratio of 7:3;
S2. Preparation of TiO₂/Fe₃O₄ doped intercalated montmorillonite modified crystal powder: 5 parts by weight of tetrabutyl titanate were added into 100 parts by weight of ethanol to form a solution A, 0.5 parts by weight of melamine, 3.4 parts by weight of ferric chloride and 1.2 parts by weight of ferrous chloride were dissolved in 70 parts by weight of water to obtain a solution B, the solution B was dropwise added into the solution A, the mixture was stirred for 10 min to obtain a pillaring agent, the pillaring agent was dropwise added into 200 parts by weight of suspension containing 30 parts by weight of montmorillonite while 10 parts by weight of 25 wt % ammonia water was dropwise added, 100 parts by weight of the surface-modified natural mineral crystal powder prepared in step S1 was added under nitrogen protection, the mixture was heated to 65° C., carried out reaction under stirring for 2 h, stood for aging for 1 h, performed microwave heating and drying at 500 W, then washed, ball milled for 1 h and calcined at 450° C. for 1 h to prepare the TiO₂/Fe₃O₄ doped intercalated montmorillonite modified crystal powder;
S3. Preparation of modified crystal powder: 20 parts by weight of the TiO₂/Fe₃O₄ doped intercalated montmorillonite modified crystal powder prepared in step S2 were added into 100 parts by weight of water, 14 parts by weight of dopamine hydrochloride and 1 part by weight of catalyst were added, the mixture was heated to 40° C., carried out reaction under stirring for 2 h, filtered, washed and dried to prepare the modified crystal powder;
the catalyst is a Tris-HCl solution with pH=8.5;
S4. Essential oil adsorption: 100 parts by weight of the modified crystal powder prepared in step S3 were added into 100 parts by weight of petroleum ether, 15 parts by weight of compound aromatic essential oil were added, and the solvent was evaporated to dryness to prepare the essential oil-adsorbed modified crystal powder;
the compound aromatic essential oil is a mixture of rose essential oil and wormwood essential oil with a mass ratio of 5:3;
S5. Preparation of biomineral crystal material: 10 parts by weight of the essential oil-adsorbed modified crystal powder prepared in step S4 were added into 200 parts by weight of water, 17 parts by weight of sodium alginate and 1 part by weight of Tween-20 were added to obtain an aqueous phase, the aqueous phase was dropwise added into 500 parts by weight of fish oil, the mixture was emulsified at 10000 r/min for 15 min, then 10 parts by weight of 3 wt % calcium chloride solution was dropwise added, the mixture was cured at room temperature for 20 min, filtered, washed and dried to prepare the biomineral crystal material.

### Example 2

This example provides a preparation method of a biomineral crystal material, which specifically includes the following steps:
S1. Surface modification of natural mineral crystal: 50 parts by weight of natural mineral crystal were ball-milled for 1.5 h, the milled crystal was added into 200 parts by weight of ethanol, 7 parts by weight of silane coupling agent was added, the mixture was heated to 50° C. and carried out reaction under stirring for 3 h, then filtered, washed and dried to prepare the surface-modified natural mineral crystal powder;
the silane coupling agent is a mixture of KH550 and KH580 with a mass ratio of 10:5;
S2. Preparation of TiO₂/Fe₃O₄ doped intercalated montmorillonite modified crystal powder: 7 parts by weight of tetrabutyl titanate were added into 100 parts by weight of ethanol to form a solution A, 1 parts by weight of melamine, 3.5 parts by weight of ferric chloride and 1.3 parts by weight of ferrous chloride were dissolved in 70 parts by weight of water to obtain a solution B, the solution B was dropwise added into the solution A, the mixture was stirred for 10 min to obtain a pillaring agent, the pillaring agent was dropwise added into 200 parts by weight of suspension containing 50 parts by weight of montmorillonite while 20 parts by weight of 28 wt % ammonia water was dropwise added, 100 parts by weight of the surface-modified natural mineral crystal powder prepared in step S1 was added under nitrogen protection, the mixture was heated to 75° C., carried out reaction under stirring for 4 h, stood for aging for 1 h, performed microwave heating and drying at 500 W, then washed, ball milled for 2 h and calcined at 550° C. for 3 h to prepare the TiO₂/Fe₃O₄ doped intercalated montmorillonite modified crystal powder;
S3. Preparation of modified crystal powder: 30 parts by weight of the TiO₂/Fe₃O₄ doped intercalated montmorillonite modified crystal powder prepared in step S2 were added into 100 parts by weight of water, 18 parts by weight of dopamine hydrochloride and 2 parts by weight of catalyst were added, the mixture was heated to 50° C., carried out reaction under stirring for 3 h, filtered, washed and dried to prepare the modified crystal powder;
the catalyst is a Tris-HCl solution with pH=9;
S4. Essential oil adsorption: 100 parts by weight of the modified crystal powder prepared in step S3 were added into 100 parts by weight of petroleum ether, 22 parts by weight of compound aromatic essential oil were added, and the solvent was evaporated to dryness to prepare the the compound aromatic essential oil is a mixture of rose essential oil and wormwood essential oil with a mass ratio of 7:4;
S5. Preparation of biomineral crystal material: 13 parts by weight of the essential oil-adsorbed modified crystal powder prepared in step S4 were added into 200 parts by weight of water, 20 parts by weight of sodium alginate and 2 part by weight of Tween-40 were added to obtain an aqueous phase, the aqueous phase was dropwise added into 500 parts by weight of fish oil, the mixture was emulsified at 10000 r/min for 15 min, then 10 parts by weight of 3 wt % calcium chloride solution was dropwise added, the mixture was cured at room temperature for 30 min, filtered, washed and dried to prepare the biomineral crystal material.

### Example 3

This example provides a preparation method of a biomineral crystal material, which specifically includes the following steps:
S1. Surface modification of natural mineral crystal: 50 parts by weight of natural mineral crystal were ball-milled for 1 h, the milled crystal was added into 200 parts by weight of ethanol, 5 parts by weight of silane coupling agent was added, the mixture was heated to 45° C. and carried out reaction under stirring for 2 h, then filtered, washed and dried to prepare the surface-modified natural mineral crystal powder;
the silane coupling agent is a mixture of KH550 and KH580 with a mass ratio of 8:4;
S2. Preparation of TiO₂/Fe₃O₄ doped intercalated montmorillonite modified crystal powder: 6 parts by weight of tetrabutyl titanate were added into 100 parts by weight of ethanol to form a solution A, 0.7 parts by weight of melamine, 3.46 parts by weight of ferric chloride and 1.26 parts by weight of ferrous chloride were dissolved in 70 parts by weight of water to obtain a solution B, the solution B was dropwise added into the solution A, the mixture was stirred for 10 min to obtain a pillaring agent, the pillaring agent was dropwise added into 200 parts by weight of suspension containing 40 parts by weight of montmorillonite while 15 parts by weight of 26 wt % ammonia water was dropwise added, 100 parts by weight of the surface-modified natural mineral crystal powder prepared in step S1 was added under nitrogen protection, the mixture was heated to 70° C., carried out reaction under stirring for 3 h, stood for aging for 1 h, performed microwave heating and drying at 500 W, then washed, ball milled for 1.5 h and calcined at 500° C. for 2 h to prepare the TiO₂/Fe₃O₄ doped intercalated montmorillonite modified crystal powder;
S3. Preparation of modified crystal powder: 25 parts by weight of the TiO₂/Fe₃O₄ doped intercalated montmorillonite modified crystal powder prepared in step S2 were added into 100 parts by weight of water, 16 parts by weight of dopamine hydrochloride and 1.5 parts by weight of catalyst were added, the mixture was heated to 45° C., carried out reaction under stirring for 2.5 the catalyst is a Tris-HCl solution with pH=8.7;
S4. Essential oil adsorption: 100 parts by weight of the modified crystal powder prepared in step S3 were added into 100 parts by weight of petroleum ether, 19 parts by weight of compound aromatic essential oil were added, and the solvent was evaporated to dryness to prepare the essential oil-adsorbed modified crystal powder;
the compound aromatic essential oil is a mixture of rose essential oil and wormwood essential oil with a mass ratio of 6:3.5;
S5. Preparation of biomineral crystal material: 11 parts by weight of the essential oil-adsorbed modified crystal powder prepared in step S4 were added into 200 parts by weight of water, 180 parts by weight of sodium alginate and 1.2 parts by weight of Tween-85 were added to obtain an aqueous phase, the aqueous phase was dropwise added into 500 parts by weight of fish oil, the mixture was emulsified at 10000 r/min for 15 min, then 10 parts by weight of 3 wt % calcium chloride solution was dropwise added, the mixture was cured at room temperature for 25 min, filtered, washed and dried to prepare the biomineral crystal material.

### Example 4

Compared with Example 3, the only difference is that the silane coupling agent is single KH550.

### Example 5

Compared with Example 3, the only difference is that the silane coupling agent is single KH580.

### Comparative Example 1

Compared with Example 3, the only difference is that step S1 is not performed.

Specific details are as follows:
S1. Preparation of TiO₂/Fe₃O₄ doped intercalated montmorillonite modified crystal powder: 6 parts by weight of tetrabutyl titanate were added into 100 parts by weight of ethanol to form a solution A, 0.7 parts by weight of melamine, 3.46 parts by weight of ferric chloride and 1.26 parts by weight of ferrous chloride were dissolved in 70 parts by weight of water to obtain a solution B, the solution B was dropwise added into the solution A, the mixture was stirred for 10 min to obtain a pillaring agent, the pillaring agent was dropwise added into 200 parts by weight of suspension containing 40 parts by weight of montmorillonite while 15 parts by weight of 26 wt % ammonia water was dropwise added, 100 parts by weight of the natural mineral crystal powder was added under nitrogen protection, the mixture was heated to 70° C., carried out reaction under stirring for 3 h, stood for aging for 1 h, performed microwave heating and drying at 500 W, then washed, ball milled for 1.5 h and calcined at 500° C. for 2 h to prepare the TiO₂/Fe₃O₄ doped intercalated montmorillonite modified crystal powder;
S2. Preparation of modified crystal powder: 25 parts by weight of the TiO₂/Fe₃O₄ doped intercalated montmorillonite modified crystal powder prepared in step S1 were added into 100 parts by weight of water, 16 parts by weight of dopamine hydrochloride and 1.5 parts by weight of catalyst were added, the mixture was heated to 45° C., carried out reaction under stirring for 2.5 h, filtered, washed and dried to prepare the modified crystal powder;
the catalyst is a Tris-HCl solution with pH=8.7;
S3. Essential oil adsorption: 100 parts by weight of the modified crystal powder prepared in step S2 were added into 100 parts by weight of petroleum ether, 19 parts by weight of compound aromatic essential oil were added, and the solvent was evaporated to dryness to prepare the essential oil-adsorbed modified crystal powder;
the compound aromatic essential oil is a mixture of rose essential oil and wormwood essential oil with a mass ratio of 6:3.5;
S4. Preparation of biomineral crystal material: 11 parts by weight of the essential oil-adsorbed modified crystal powder prepared in step S3 were added into 200 parts by weight of water, 180 parts by weight of sodium alginate and 1.2 parts by weight of Tween-85 were added to obtain an aqueous phase, the aqueous phase was dropwise added into 500 parts by weight of fish oil, the mixture was emulsified at 10000 r/min for 15 min, then 10 parts by weight of 3 wt % calcium chloride solution was dropwise added, the mixture was cured at room temperature for 25 min, filtered, washed and dried to prepare the biomineral crystal material.

### Comparative Example 2

Compared with Example 3, the only difference is that no melamine is added in step S2.

Specific details are as follows:
S2. Preparation of TiO₂/Fe₃O₄ doped intercalated montmorillonite modified crystal powder: 6 parts by weight of tetrabutyl titanate were added into 100 parts by weight of ethanol to form a solution A, 3.46 parts by weight of ferric chloride and 1.26 parts by weight of ferrous chloride were dissolved in 70 parts by weight of water to obtain a solution B, the solution B was dropwise added into the solution A, the mixture was stirred for 10 min to obtain a pillaring agent, the pillaring agent was dropwise added into 200 parts by weight of suspension containing 40 parts by weight of montmorillonite while 15 parts by weight of 26 wt % ammonia water was dropwise added, 100 parts by weight of the surface-modified natural mineral crystal powder prepared in step S1 was added under nitrogen protection, the mixture was heated to 70° C., carried out reaction under stirring for 3 h, stood for aging for 1 h, performed microwave heating and drying at 500 W, then washed, ball milled for 1.5 h and calcined at 500° C. for 2 h to prepare the TiO₂/Fe₃O₄ intercalated montmorillonite modified crystal powder.

### Comparative Example 3

Compared with Example 3, the only difference is that no ferric chloride or ferrous chloride is added in step S2.

Specific details are as follows:
S2. Preparation of TiO₂ doped intercalated montmorillonite modified crystal powder: 10.72 parts by weight of tetrabutyl titanate were added into 100 parts by weight of ethanol to form a solution A, 0.7 parts by weight of melamine were dissolved in 70 parts by weight of water to obtain a solution B, the solution B was dropwise added into the solution A, the mixture was stirred for 10 min to obtain a pillaring agent, the pillaring agent was dropwise added into 200 parts by weight of suspension containing 40 parts by weight of montmorillonite while 15 parts by weight of 26 wt % ammonia water was dropwise added, 100 parts by weight of the surface-modified natural mineral crystal powder prepared in step S1 was added under nitrogen protection, the mixture was heated to 70° C., carried out reaction under stirring for 3 h, stood for aging for 1 h, performed microwave heating and drying at 500 W, then washed, ball milled for 1.5 h and calcined at 500° C. for 2 h to prepare the TiO₂ doped intercalated montmorillonite modified crystal powder.

### Comparative Example 4

Compared with Example 3, the only difference is that step S2 is not performed.

Specific details are as follows:
S1. Surface modification of natural mineral crystal: 50 parts by weight of natural mineral crystal were ball-milled for 1 h, the milled crystal was added into 200 parts by weight of ethanol, 5 parts by weight of silane coupling agent was added, the mixture was heated to 45° C. and carried out reaction under stirring for 2 h, then filtered, washed and dried to prepare the surface-modified natural mineral crystal powder;
the silane coupling agent is a mixture of KH550 and KH580 with a mass ratio of 8:4;
S2. Preparation of modified crystal powder: 25 parts by weight of the surface-modified natural mineral crystal powder prepared in step S1 were added into 100 parts by weight of water, 16 parts by weight of dopamine hydrochloride and 1.5 parts by weight of catalyst were added, the mixture was heated to 45° C., carried out reaction under stirring for 2.5 h, filtered, washed and dried to prepare the modified crystal powder;
the catalyst is a Tris-HCl solution with pH=8.7;
S3. Essential oil adsorption: 100 parts by weight of the modified crystal powder prepared in step S2 were added into 100 parts by weight of petroleum ether, 19 parts by weight of compound aromatic essential oil were added, and the solvent was evaporated to dryness to prepare the the compound aromatic essential oil is a mixture of rose essential oil and wormwood essential oil with a mass ratio of 6:3.5;
S4. Preparation of biomineral crystal material: 11 parts by weight of the essential oil-adsorbed modified crystal powder prepared in step S3 were added into 200 parts by weight of water, 180 parts by weight of sodium alginate and 1.2 parts by weight of Tween-85 were added to obtain an aqueous phase, the aqueous phase was dropwise added into 500 parts by weight of fish oil, the mixture was emulsified at 10000 r/min for 15 min, then 10 parts by weight of 3 wt % calcium chloride solution was dropwise added, the mixture was cured at room temperature for 25 min, filtered, washed and dried to prepare the biomineral crystal material.

### Comparative Example 5

Compared with Example 3, the only difference is that step S3 is not performed.

Specific details are as follows:
S1. Surface modification of natural mineral crystal: 50 parts by weight of natural mineral crystal were ball-milled for 1 h, the milled crystal was added into 200 parts by weight of ethanol, 5 parts by weight of silane coupling agent was added, the mixture was heated to 45° C. and carried out reaction under stirring for 2 h, then filtered, washed and dried to prepare the surface-modified natural mineral crystal powder;
the silane coupling agent is a mixture of KH550 and KH580 with a mass ratio of 8:4;
S2. Preparation of TiO₂/Fe₃O₄ doped intercalated montmorillonite modified crystal powder: 6 parts by weight of tetrabutyl titanate were added into 100 parts by weight of ethanol to form a solution A, 0.7 parts by weight of melamine, 3.46 parts by weight of ferric chloride and 1.26 parts by weight of ferrous chloride were dissolved in 70 parts by weight of water to obtain a solution B, the solution B was dropwise added into the solution A, the mixture was stirred for 10 min to obtain a pillaring agent, the pillaring agent was dropwise added into 200 parts by weight of suspension containing 40 parts by weight of montmorillonite while 15 parts by weight of 26 wt % ammonia water was dropwise added, 100 parts by weight of the surface-modified natural mineral crystal powder prepared in step S1 was added under nitrogen protection, the mixture was heated to 70° C., carried out reaction under stirring for 3 h, stood for aging for 1 h, performed microwave heating and drying at 500 W, then washed, ball milled for 1.5 h and calcined at 500° C. for 2 h to prepare the TiO₂/Fe₃O₄ doped intercalated montmorillonite modified crystal powder;
S3. Essential oil adsorption: 100 parts by weight of the TiO₂/Fe₃O₄ doped intercalated montmorillonite modified crystal powder prepared in step S2 were added into 100 parts by weight of petroleum ether, 19 parts by weight of compound aromatic essential oil were added, and the solvent was evaporated to dryness to prepare the essential oil-adsorbed modified crystal powder;
the compound aromatic essential oil is a mixture of rose essential oil and wormwood essential oil with a mass ratio of 6:3.5;
S4. Preparation of biomineral crystal material: 11 parts by weight of the essential oil-adsorbed modified crystal powder prepared in step S3 were added into 200 parts by weight of water, 180 parts by weight of sodium alginate and 1.2 parts by weight of Tween-85 were added to obtain an aqueous phase, the aqueous phase was dropwise added into 500 parts by weight of fish oil, the mixture was emulsified at 10000 r/min for 15 min, then 10 parts by weight of 3 wt % calcium chloride solution was dropwise added, the mixture was cured at room temperature for 25 min, filtered, washed and dried to prepare the biomineral crystal material.

### Comparative Example 6

Compared with Example 3, the only difference is that step S4 is not performed.

Specific details are as follows:
S1. Surface modification of natural mineral crystal: 50 parts by weight of natural mineral crystal were ball-milled for 1 h, the milled crystal was added into 200 parts by weight of ethanol, 5 parts by weight of silane coupling agent was added, the mixture was heated to 45° C. and carried out reaction under stirring for 2 h, then filtered, washed and dried to prepare the surface-modified natural mineral crystal powder;
the silane coupling agent is a mixture of KH550 and KH580 with a mass ratio of 8:4;
S2. Preparation of TiO₂/Fe₃O₄ doped intercalated montmorillonite modified crystal powder: 6 parts by weight of tetrabutyl titanate were added into 100 parts by weight of ethanol to form a solution A, 0.7 parts by weight of melamine, 3.46 parts by weight of ferric chloride and 1.26 parts by weight of ferrous chloride were dissolved in 70 parts by weight of water to obtain a solution B, the solution B was dropwise added into the solution A, the mixture was stirred for 10 min to obtain a pillaring agent, the pillaring agent was dropwise added into 200 parts by weight of suspension containing 40 parts by weight of montmorillonite while 15 parts by weight of 26 wt % ammonia water was dropwise added, 100 parts by weight of the surface-modified natural mineral crystal powder prepared in step S1 was added under nitrogen protection, the mixture was heated to 70° C., carried out reaction under stirring for 3 h, stood for aging for 1 h, performed microwave heating and drying at 500 W, then washed, ball milled for 1.5 h and calcined at 500° C. for 2 h to prepare the TiO₂/Fe₃O₄ doped intercalated montmorillonite modified crystal powder;
S3. Preparation of modified crystal powder: 25 parts by weight of the TiO₂/Fe₃O₄ doped intercalated montmorillonite modified crystal powder prepared in step S2 were added into 100 parts by weight of water, 16 parts by weight of dopamine hydrochloride and 1.5 parts by weight of catalyst were added, the mixture was heated to 45° C., carried out reaction under stirring for 2.5 the catalyst is a Tris-HCl solution with pH=8.7;
S4. Preparation of biomineral crystal material: 11 parts by weight of the modified crystal powder prepared in step S3 were added into 200 parts by weight of water, 180 parts by weight of sodium alginate and 1.2 parts by weight of Tween-85 were added to obtain an aqueous phase, the aqueous phase was dropwise added into 500 parts by weight of fish oil, the mixture was emulsified at 10000 r/min for 15 min, then 10 parts by weight of 3 wt % calcium chloride solution was dropwise added, the mixture was cured at room temperature for 25 min, filtered, washed and dried to prepare the biomineral crystal material.

### Comparative Example 7

Compared with Example 3, the only difference is that step S5 is not performed.

Specific details are as follows:
S1. Surface modification of natural mineral crystal: 50 parts by weight of natural mineral crystal were ball-milled for 1 h, the milled crystal was added into 200 parts by weight of ethanol, 5 parts by weight of silane coupling agent was added, the mixture was heated to 45° C. and carried out reaction under stirring for 2 h, then filtered, washed and dried to prepare the surface-modified natural mineral crystal powder;
the silane coupling agent is a mixture of KH550 and KH580 with a mass ratio of 8:4;
S2. Preparation of TiO₂/Fe₃O₄ doped intercalated montmorillonite modified crystal powder: 6 parts by weight of tetrabutyl titanate were added into 100 parts by weight of ethanol to form a solution A, 0.7 parts by weight of melamine, 3.46 parts by weight of ferric chloride and 1.26 parts by weight of ferrous chloride were dissolved in 70 parts by weight of water to obtain a solution B, the solution B was dropwise added into the solution A, the mixture was stirred for 10 min to obtain a pillaring agent, the pillaring agent was dropwise added into 200 parts by weight of suspension containing 40 parts by weight of montmorillonite while 15 parts by weight of 26 wt % ammonia water was dropwise added, 100 parts by weight of the surface-modified natural mineral crystal powder prepared in step S1 was added under nitrogen protection, the mixture was heated to 70° C., carried out reaction under stirring for 3 h, stood for aging for 1 h, performed microwave heating and drying at 500 W, then washed, ball milled for 1.5 h and calcined at 500° C. for 2 h to prepare the TiO₂/Fe₃O₄ doped intercalated montmorillonite modified crystal powder;
S3. Preparation of modified crystal powder: 25 parts by weight of the TiO₂/Fe₃O₄ doped intercalated montmorillonite modified crystal powder prepared in step S2 were added into 100 parts by weight of water, 16 parts by weight of dopamine hydrochloride and 1.5 parts by weight of catalyst were added, the mixture was heated to 45° C., carried out reaction under stirring for 2.5
the catalyst is a Tris-HCl solution with pH=8.7;
S4. Essential oil adsorption: 100 parts by weight of the modified crystal powder prepared in step S3 were added into 100 parts by weight of petroleum ether, 19 parts by weight of compound aromatic essential oil were added, and the solvent was evaporated to dryness to prepare the essential oil-adsorbed modified crystal powder, which is the biomineral crystal material;
the compound aromatic essential oil is a mixture of rose essential oil and wormwood essential oil with a mass ratio of 6:3.5.

### Test Example 1

The infrared emissivity of the biomineral crystal materials prepared in Examples 1-5 and Comparative Examples 1-7 of this disclosure in the dual bands of 3 μm-5 μm and 8 μm-14 μm at room temperature was measured by an IR-2 dual-band emissivity emissometer. The results are as shown in Table 1.

TABLE 1

| | Infrared | Infrared |
|---|---|---|
| | Emissivity in | Emissivity in |
| | 3 μm-5 μm | 8 μm-14 μm |
| Group | Band | Band |
| Example 1 | 0.65 | 0.94 |
| Example 2 | 0.63 | 0.93 |
| Example 3 | 0.66 | 0.95 |
| Example 4 | 0.61 | 0.87 |
| Example 5 | 0.59 | 0.88 |
| Comparative | 0.56 | 0.83 |
| Example 1 | | |
| Comparative | 0.58 | 0.90 |
| Example 2 | | |
| Comparative | 0.51 | 0.80 |
| Example 3 | | |
| Comparative | 0.48 | 0.75 |
| Example 4 | | |
| Comparative | 0.62 | 0.92 |
| Example 5 | | |
| Comparative | 0.61 | 0.91 |
| Example 6 | | |
| Comparative | 0.60 | 0.89 |
| Example 7 | | |

It can be seen from the table above that the biomineral crystal materials prepared in Examples 1-3 of this disclosure have high infrared emissivity in the dual bands of 3 μm-5 μm and 8 μm-14 μm.

### Test Example 2

The specific surface area and pore size distribution of the products prepared in step S2 of Examples 1-5 and Comparative Examples 1-3 of this disclosure, as well as the product prepared in step S1 of Comparative Example 4, were measured by a TriStar II 3020 specific surface area and porosity analyzer. The test results are as shown in Table 2.

TABLE 2

| | Specific | |
|---|---|---|
| | surface | Pore |
| | area | volume |
| Group | (m²/g) | (cm³/g) |
| Example 1 | 66.5 | 22.1 |
| Example 2 | 66.2 | 21.9 |
| Example 3 | 66.8 | 22.5 |
| Example 4 | 61.2 | 18.2 |
| Example 5 | 60.8 | 17.9 |
| Comparative | 56.6 | 15.7 |
| Example 1 | | |
| Comparative | 65.8 | 21.2 |
| Example 2 | | |
| Comparative | 52.6 | 13.2 |
| Example 3 | | |
| Comparative | 47.3 | 11.6 |
| Example 4 | | |

It can be seen from the table above that the products prepared in step S2 of Examples 1-3 of this disclosure have large specific surface area and pore volume.

### Test Example 3

130 SPF-grade Kunming mice were randomly divided into a blank group, Examples 1-5 groups and Comparative Examples 1-7 groups, with 10 mice in each group. During the feeding period, the mice had free access to food and water, the feeding temperature was 25±2° C., and the humidity was 50±10%. After 1 week of acclimation feeding, the biomineral crystal materials prepared in Examples 1-5 and Comparative Examples 1-7 were mixed with cotton at a mass ratio of 2:8, and the mixture was filled into non-woven bags to prepare pillows. The blank group was provided with a non-woven bag filled with pure cotton as the pillow. The prepared sleep-aiding pillow or control pillow was laid on a top cover of a mouse cage, and the top cover was kept fastened during the test. On the 15th day of the test, the mice were put into a mouse spontaneous activity monitor in batches, accumulated for 5 minutes, and then the number of spontaneous activities in mice within 5 minutes was recorded. The number of spontaneous activities in mice of the animal groups in the test groups and the control group were compared, and the results are as shown in Table 3.

TABLE 3

| | Number of |
|---|---|
| | activities |
| Group | (times/5 min) |
| Blank group | 172.2 ± 24.1 |
| Example 1 | 131.4 ± 19.5* |
| Example 2 | 134.3 ± 22.6* |
| Example 3 | 126.7 ± 21.4* |
| Example 4 | 156.3 ± 31.6 |
| Example 5 | 158.2 ± 28.8 |
| Comparative | 161.4 ± 26.4 |
| Example 1 | |
| Comparative | 160.8 ± 24.7 |
| Example 2 | |
| Comparative | 164.2 ± 28.1 |
| Example 3 | |
| Comparative | 166.9 ± 30.4 |
| Example 4 | |
| Comparative | 162.4 ± 27.9 |
| Example 5 | |
| Comparative | 167.6 ± 32.2 |
| Example 6 | |
| Comparative | 163.8 ± 29.5 |
| Example 7 | |

Note:
*means P < 0.05 compared with the blank group.

It can be seen from the table above that the pillows filled with the biomineral crystal materials prepared in Examples 1-3 of this disclosure can significantly reduce the number of activities in mice and help mice fall asleep.

The above are only preferred embodiments of this disclosure, and are not intended to limit this disclosure. Any modification, equivalent replacement, improvement, etc., made within the spirit and principles of the present disclosure shall fall within the scope of protection of this disclosure.

What is claimed is:

1. A preparation method of a biomineral crystal material, comprising the following steps:
S1. surface modification of natural mineral crystal: ball-milling natural mineral crystal, adding the milled crystal into ethanol, adding a silane coupling agent, heating the mixture and carrying out reaction under stirring, filtering, washing, and drying to prepare surface-modified natural mineral crystal powder; a mass ratio of the natural mineral crystal to the silane coupling agent is 50:4-7, the silane coupling agent is a mixture of KH550 and KH580 with a mass ratio of 7-10:3-5; the natural mineral crystal is a mixed ore containing natural mineral crystal and rare earth concentrate with a mass ratio of 10:5;
S2. preparation of TiO₂/Fe₃O₄ doped intercalated montmorillonite modified crystal powder: adding tetrabutyl titanate into ethanol to form a solution A, dissolving melamine, ferric chloride and ferrous chloride in water to obtain a solution B, dropwise adding the solution B into the solution A, stirring evenly to obtain a pillaring agent, dropwise adding the pillaring agent into a montmorillonite suspension while dropwise adding ammonia water, adding the surface-modified natural mineral crystal powder prepared in step S1, heating the mixture and carrying out reaction under stirring, standing for aging, and performing microwave heating and drying, washing, ball milling and calcination to prepare the TiO₂/Fe₃O₄ doped intercalated montmorillonite modified crystal powder;
S3. preparation of modified crystal powder: adding the TiO₂/Fe₃O₄ doped intercalated montmorillonite modified crystal powder prepared in step S2 into water, adding dopamine hydrochloride and a catalyst, heating the mixture and carrying out reaction under stirring, filtering, washing, and drying to prepare the modified crystal powder;
S4. essential oil adsorption: adding the modified crystal powder prepared in step S3 into petroleum ether, adding compound aromatic essential oil, and evaporating the solvent to dryness to prepare essential oil-adsorbed modified crystal powder; and
S5. preparation of biomineral crystal material: adding the essential oil-adsorbed modified crystal powder prepared in step S4 into water, adding sodium alginate and an emulsifier to obtain an aqueous phase, dropwise adding the aqueous phase into fish oil for emulsification, then dropwise adding a calcium chloride solution, performing room temperature curing, filtering, washing and drying to prepare the biomineral crystal material.

2. The preparation method according to claim 1, wherein in step S1, the ball milling is performed for 0.5-1.5 h, and the mixture is heated and carried out reaction under stirring at 40-50° C. for 1-3 h.

3. The preparation method according to claim 1, wherein in step S2, a mass ratio of tetrabutyl titanate, melamine, ferric chloride, ferrous chloride, montmorillonite to the surface-modified natural mineral crystal powder is 5-7:0.5-1:3.4-3.5:1.2-1.3:30-50:100, the mixture is heated and carried out reaction under stirring at 65-75° C. for 2-4 h, the ball milling is performed for 1-2 h, and the calcination is performed at 450-550° C. for 1-3 h.

4. The preparation method according to claim 1, wherein in step S3, a mass ratio of the TiO₂/Fe₃O₄ doped intercalated montmorillonite modified crystal powder, the dopamine hydrochloride to the catalyst is 20-30:14-18:1-2, the mixture is heated and carried out reaction under stirring at 40-50° C. for 2-3 h, and the catalyst is a Tris-HCl solution with pH=8.5-9.

5. The preparation method according to claim 1, wherein in step S4, a mass ratio of the modified crystal powder to a compound aromatic essential oil is 100:15-22, the compound aromatic essential oil is at least two selected from a group consisting of rose essential oil, clove essential oil, lavender essential oil and wormwood essential oil, and the essential oil is prepared by subjecting raw materials to supercritical fluid extraction.

6. The preparation method according to claim 1, wherein in step S5, a mass ratio of the essential oil-adsorbed modified crystal powder, the sodium alginate to the emulsifier is 10-13:17-20:1-2, the emulsifier is at least one selected from a group consisting of Tween-20, Tween-40, Tween-60, Tween-80 and Tween-85, and the room temperature curing is performed for 20-30 min.

7. A biomineral crystal material prepared by the preparation method according to claim 1.

8. An application of the biomineral crystal material according to claim 7 in preparing sleep-aiding products.
